(19) United States
(12) Patent Application Publication
(10) Pub. No.: US 2024/0350432 A1
(43) Pub. Date: Oct. 24, 2024

(54) CENTRAL NERVOUS SYSTEM MODULATORS AS COVID-19 THERAPEUTICS

(71) Applicants: Bobbi Jo Mullins, Ingleside, IL (US); University of Kentucky Research Foundation, Lexington, KY (US)

(72) Inventors: Jill Rebecca Turner Ortinski, Lexington, KY (US); Linda P. Dwoskin, Lexington, KY (US); Bobbie Jo Mullins, Ingleside, IL (US)

(21) Appl. No.: 18/640,714

(22) Filed: Apr. 19, 2024

Related U.S. Application Data
(60) Provisional application No. 63/497,313, filed on Apr. 20, 2023.

(51) Int. Cl. A61K 31/13; A61K 9/00; A61K 31/44; A61K 31/465; A61K 31/573; A61K 45/06; A61P 31/12

(52) U.S. Cl. CPC A61K 31/13; A61K 9/0019; A61K 31/44; A61K 31/465; A61K 31/573; A61K 45/06; A61P 31/12

(57) ABSTRACT

This disclosure relates to methods for the prevention and/or treatment of neurological adverse effects caused by viral infections in a subject. In some aspects, the methods include administration of at least one N-methyl-D-aspartic acid receptor (NMDAR) antagonist to a subject as a preventative treatment. In other aspects, the NMDAR antagonists may be used as a treatment following a viral infection or suspected viral infection.

### CROSS-REFERENCE TO RELATED APPLICATIONS

This disclosure claims priority to U.S. Provisional Patent Application 63/497,313, filed Apr. 20, 2023, which is hereby incorporated by reference in its entirety.

### GOVERNMENT SUPPORT

This application was made with support from the National Institute of General Medical Sciences under grant UL1TR001998. The Government may have certain rights to the invention.

### FIELD

This disclosure relates to methods for the prevention and/or treatment of neurological pathologies caused by viral infections in a subject.

### BACKGROUND

Neuro-invasiveness and/or neurological side effects can be fatal adverse effects of a viral infection in subjects. While a virus infection itself can be difficult to target pharmacologically, targeting the cellular mechanisms that can be disrupted or impacted can help to prevent or treat the subject and reduce the overall impact on the subject's health.

One of the more challenging pathologies to treat from a viral infection is when the virus penetrates the central nervous system (CNS). For example, pathological studies from the SARS-CoV-2 coronavirus provide recent evidence that suggests that the respiratory symptoms of CoV-2 disease (COVID-19) may be related to effects of the virus on the CNS. A report by Mao et al. (JAMA Neurol. 2020 77:683-690) found that 36.4% of SARS-CoV-2 (COVID-19) infected patients suffered neurological symptoms, including loss of smell/taste, sensation of having to consciously think about breathing, headache, seizures, nausea and vomiting. Moreover, patients with more severe respiratory symptoms were 50% more likely to display these neurological symptoms, compared to those with less severe pneumonia-like symptoms. To exert these effects, SARS-CoV-2 invades the central nervous system (CNS) using various methods⁶⁻⁸. The virus is known to use receptors such as ACE2 or NRP1⁹⁻¹³, which are highly expressed in the lungs and upper respiratory tract^{14,15}, to enter cells. The lungs and upper respiratory tract are innervated by the vagus nerve which travels to the brainstem allowing SARS-CoV-2 entry to the respiratory centers of the brain^{10,17,18}. Notably, the medulla oblongata and pons, located in the brainstem, regulate respiration¹⁹. There is evidence to suggest that once infiltrated into the brain, SARS-CoV-2 induces cytokine release in the CNS, resulting in activation of microglia²⁰. Among other homeostatic functions, microglia are resident immune cells that engulf foreign substances invading the CNS^{21,22}. Astrocytes are also resident CNS cells important for blood brain barrier integrity and physiologic functions^{23,24}. Interestingly, microglia are known to induce astrocyte reactivity^{25,26}, which precipitates demyelination of axons¹⁷ and additional cytokine release²⁷. These cells exhibit morphological differences when activated, making them reliable markers for neuroinflammation^{21,24}. Given that COVID-19 is a respiratory illness, the respiratory centers of the brain are of interest, specifically as they relate to immune response to CNS invasion. It is hypothesized that SARS-CoV-2 invades these respiratory centers, leading to neuronal damage characterized by visible demyelination, morphological alterations, increased neuronal death, and heightened pro-inflammatory markers such as astrocytes and microglia.

Neuroinflammation is a multi-faceted condition, with many known causes but limited treatments. N-methyl-D-aspartate (NMDA) receptor antagonists are a medication class known to have neuroprotective effects in environments with excitotoxicity, where the overactivation of NMDA receptors causes cell death, and may also have a preventative effect on neuroinflammatory processes²⁸⁻³⁰. Examples of NMDA receptor antagonists include memantine, which is a wide-spread inhibitor that exhibits off-target effects³¹, and ifenprodil, which is a more specific inhibitor to the receptor subunit most closely associated with excitotoxicity³²⁻³⁴. Excessive neuroinflammation in the pons and medulla due to COVID-19 exposure may respond to NMDA receptor antagonism when murine subjects are pretreated with memantine or ifenprodil. Thus, while the present study primarily aims to evaluate if COVID-19 exposure induces neuroinflammation in the respiratory centers of the brain, a secondary aim is to elucidate the role NMDA receptor antagonists play in the pro-inflammatory process as well as to assess the extent to which these agents offer neuroprotective measures in the context of COVID-19. Murine subjects pre-treated with a NMDA receptor antagonist and inoculated with SARS-CoV-2 were examined over a time course of disease progression. Several markers of neuropathology were investigated, including demyelination and neurodegeneration, microglia, and reactive astrocytes, as well as general neuronal morphology, with primary focus on the respiratory centers of the brain, namely the pons and medulla oblongata.

As such, there is a need to lessen the impact of the viral infection on the central nervous system. The present disclosure concerns a novel approach to address the need through treating pathways negatively impacted by the virus, either as a form of prophylaxis or as treatment following the onset of a positive infection or the onset of symptoms.

### SUMMARY

The present disclosure concerns methods for preventing and/or treating neural pathologies caused by or incidental to a viral infection. In some aspects, the methods include administrations of at least one N-methyl-D-aspartic acid receptor (NMDAR) antagonist to a subject, such as a human subject.

A 1^{st} aspect of the present disclosure, either alone or in combination with any other aspect as set forth herein, concerns a method for preventing a viral induced neuropathology in a subject comprising prophylactic administration of at least one N-methyl-D-aspartic acid receptor (NMDAR) antagonist to the subject.

A 2^{nd} aspect of the present disclosure, either alone or in combination with any other aspect as set forth herein, concerns the method of the 1^{st} aspect, wherein the at least one NMDAR antagonist is selected from memantine, ketamine, ifenprodil, donepezil, amantadine, atomoxetine, agmatine, dextrallorphan, dextromethorphan, dextrophan, dizocilpine, neramexane, remacemide, eliprodil, selfotel, or combinations thereof.

A 4^{th} aspect of the present disclosure, either alone or in combination with any other aspect as set forth herein, concerns the method of the 2^{nd} or 3^{rd} aspect, wherein the at least one NMDAR includes memantine.

A 5^{th} aspect of the present disclosure, either alone or in combination with any other aspect as set forth herein, concerns the method of the 2^{nd} or 3^{rd} aspect, further comprising administration with an anti-viral therapy.

A 6^{th} aspect of the present disclosure, either alone or in combination with any other aspect as set forth herein, concerns the method of the 2^{nd} or 3^{rd} aspect, further comprising administration with a systemic steroid selected from the group consisting of dexamethasone, hydrocortisone, methylprednisolone, prednisone, or combinations thereof.

A 7^{th} aspect of the present disclosure, either alone or in combination with any other aspect as set forth herein, concerns the method of the 2^{nd} or 3^{rd} aspect, further comprising administration with nicotine.

An 8^{th} aspect of the present disclosure, either alone or in combination with any other aspect as set forth herein, concerns the method of the 2^{nd} or 3^{rd} aspect, wherein the viral induced neuropathology is caused by a measles virus, a coronavirus, an enterovirus, a adenovirus, an arbovirus, an Arenavirdae, a Bornavirdae, a Flavivirdae, a Hepadnavirdae, a Herpesvirdae, human immunodeficiency virus, human T-lymphotropic virus type I, a paramyxovirdae, a Picornavirdae, a Rhabdovirdae, a Togavirdae, and/or an influenza virus.

A 9^{th} aspect of the present disclosure, either alone or in combination with any other aspect as set forth herein, concerns the method of the 8^{th} aspect, wherein the virus is SARS-CoV-1, MERS-CoV, and/or SARS-CoV-2 and/or its variants.

A 10^{th} aspect of the present disclosure, either alone or in combination with any other aspect as set forth herein, concerns the method of the 2^{nd} or 3^{rd} aspect, wherein the subject is vulnerable to a viral infection.

An 11^{th} aspect of the present disclosure, either alone or in combination with any other aspect as set forth herein, concerns the method of the 2^{nd} or 3^{rd} aspect, wherein the at least one NMDAR antagonist is administered orally, intravenously, by infusion, transdermally, sublingually, intramuscularly, by inhalation, rectally, vaginally, subcutaneously, opthalmically, buccally, nasally, enterally, topically, intrathecally, or combinations thereof.

A 12^{th} aspect of the present disclosure, either alone or in combination with any other aspect as set forth herein, concerns the method of the 2^{nd} or 3^{rd} aspect, wherein the NMDAR antagonist is administered with a vehicle, an excipient, and/or a pharmaceutically acceptable carrier.

A 13^{th} aspect of the present disclosure, either alone or in combination with any other aspect as set forth herein, concerns a method for treating a viral induced neuropathology in a subject comprising prophylactic administration of at least one N-methyl-D-aspartic acid receptor (NMDAR) antagonist to the subject.

A 14^{th} aspect of the present disclosure, either alone or in combination with any other aspect as set forth herein, concerns the method of the 13^{th} aspect, wherein the at least one NMDAR antagonist is selected from memantine, ketamine, ifenprodil, donepezil, amantadine, atomoxetine, agmatine, dextrallorphan, dextromethorphan, dextrophan, dizocilpine, neramexane, remacemide, eliprodil, selfotel, or combinations thereof.

A 15^{th} aspect of the present disclosure, either alone or in combination with any other aspect as set forth herein, concerns the method of the 13^{th} aspect, wherein the at least one NMDAR includes memantine.

A 16^{th} aspect of the present disclosure, either alone or in combination with any other aspect as set forth herein, concerns the method of the 14^{th} or 15^{th} aspect, further comprising administration with a nAChR antagonist, an anti-nicotinic, an anti-cholinergic, or a combination thereof.

A 17^{th} aspect of the present disclosure, either alone or in combination with any other aspect as set forth herein, concerns the method of the 14^{th} or 15^{th} aspect, further comprising administration with an anti-viral therapy.

An 18^{th} aspect of the present disclosure, either alone or in combination with any other aspect as set forth herein, concerns the method of the 14^{th} or 15^{th} aspect, further comprising administration with a systemic steroid selected from the group consisting of dexamethasone, hydrocortisone, methylprednisolone, prednisone, or combinations thereof.

A 19^{th} aspect of the present disclosure, either alone or in combination with any other aspect as set forth herein, concerns the method of the 14^{th} or 15^{th} aspect, wherein the viral induced neuropathology is caused by a measles virus, a coronavirus, an enterovirus, a adenovirus, an arbovirus, an Arenavirdae, a Bornavirdae, a Flavivirdae, a Hepadnavirdae, a Herpesvirdae, human immunodeficiency virus, human T-lymphotropic virus type I, a paramyxovirdae, a Picornavirdae, a Rhabdovirdae, a Togavirdae, and/or an influenza virus.

A 20^{th} aspect of the present disclosure, either alone or in combination with any other aspect as set forth herein, concerns the method of the 19^{th} aspect, wherein the virus is SARS-CoV-1, MERS-CoV, and/or SARS-CoV-2 and/or its variants.

A 21^{st} aspect of the present disclosure, either alone or in combination with any other aspect as set forth herein, concerns the method of the 14^{th} or 15^{th} aspect, wherein the subject is vulnerable to a viral infection.

A 22^{nd} aspect of the present disclosure, either alone or in combination with any other aspect as set forth herein, concerns the method of the 14^{th} or 15^{th} aspect, wherein the at least one NMDAR antagonist is administered orally, intravenously, by infusion, transdermally, sublingually, intramuscularly, by inhalation, rectally, vaginally, subcutaneously, opthalmically, buccally, nasally, enterally, topically, intrathecally, or combinations thereof.

A 23^{rd} aspect of the present disclosure, either alone or in combination with any other aspect as set forth herein, concerns the method of the 14^{th} or 15^{th} aspect, wherein the NMDAR antagonist is administered with a vehicle, an excipient, and/or a pharmaceutically acceptable carrier.

### BRIEF DESCRIPTION OF THE DRAWINGS

FIG. 1 shows an overview of the experimental design. In a mouse model susceptible to SARS-CoV-2, animals were divided into 12 experimental groups. Animals inoculated with either SARS-CoV-2 or vehicle were pre-treated twice daily (BID) with either saline, memantine, or ifenprodil, and sacrificed at either 3, 6, or 10 days post-inoculation.

FIG. 2 shows an summary of survival and health outcomes. FIG. 2a shows survival outcomes of Groups 1, 4, 8, and 12 ((−) Control, Saline, Memantine, and Ifenprodil at 10 Days, respectively). **p<0.01 (compared to (−) Control) and #p<0.05 (compared to Saline). FIG. 2b shows health outcomes of Groups 1, 4, 8, and 12. Body weight, as a measure of disease incidence, is graphed as % baseline weight over time. Non-linear regression analyses for (−) Control compared to FIG. 2c Saline, FIG. 2d Memantine, and FIG. 2e Ifenprodil treated groups are also shown as separate graphs. Dotted lines represent 95% CI.

FIG. 3 shows Cresyl Violet staining. FIG. 3a shows visualization of morphological differences in medulla samples among key groups using cresyl violet staining. Key groups include those sacrificed 10 days after COVID-19 inoculation, with representative images of samples either positive or negative for COVID-19 and treated with saline (control), memantine, or ifenprodil. Each image is shown in the 4× and 10× magnification with anatomical markers labeled. FIG. 3b shows examples of images representative of each semi-qualitative rank for Cresyl Violet stain. FIG. 3c shows Cresyl violet (CV) qualitative average rankings according to time sacrificed post-virus inoculation and treatment group. Data points are representative of sections in pons and medulla. Error bars represent average qualitative ranking+/−S.E.M. Significant differences between groups are denoted with *p<0.05, **p<0.01, ***p<0.005, and ****p<0.001. N=3-20 sections per group.

FIG. 4 shows Black-Gold Myelin staining. FIG. 4a shows visualization of myelination in samples of medulla among key groups using black-gold II staining. Key groups include those sacrificed 10 days after COVID-19 inoculation, with representative images of samples either positive or negative for COVID-19 and treated with saline (control), memantine, or ifenprodil. Each image is shown in the 4× and 10× magnification with anatomical markers labeled. FIG. 4b shows examples of images representative of each semi-qualitative rank for Black-Gold II stain. FIG. 4c shows Black-Gold II Myelin qualitative average rankings according to time sacrificed post-virus inoculation and treatment group. Data points are representative of sections in pons and medulla. Error bars represent average qualitative ranking+/−S.E.M. Significant differences between groups are denoted with *p<0.05, **p<0.01, ***p<0.005, and ****p<0.001. N=5-18 sections per group.

FIG. 5 shows GFAP fluorescent (FL) Intensity. Data points are representative of sections in pons and medulla. FIG. 5a shows images of sections representing immunofluorescent staining for GFAP from the medulla of a COVID-19 negative brain and COVID-19 positive brain from each key treatment group. Key groups include those sacrificed 10 days post-inoculation. FIG. 5b shows GFAP FL intensity according to drug treatment. All animals were sacrificed 3 days post-virus inoculation. N=2-14 sections per group. FIG. 5c shows GFAP FL intensity according to drug treatment. All animals were sacrificed 6 days post-virus inoculation. N=11-15 sections per group. FIG. 5d shows GFAP FL intensity according to positive or negative COVID-19 status and drug treatment. All animals were sacrificed 10 days post-virus inoculation. Results reported as mean FL intensity+/−S.E.M. with *p<0.05 and **p<0.01. N=5-18 sections per group. FIG. 5e shows GFAP FL intensity nonlinear fit based on time post-inoculation for each COVID-19 positive treatment group (saline, memantine, or ifenprodil). Dotted lines represent 95% CI. The graph's upper left-hand corner lists the slope+/−95% CI, R (correlation coefficient), F statistics, and p-value. N=2-15 sections per group.

FIG. 6 shows IBA1 fluorescent (FL) Intensity. Data points are representative of sections in pons and medulla. FIG. 6a shows images of sections representing immunofluorescent staining for IBA1 from the medulla of a COVID-19 negative brain and COVID-19 positive brain from each key treatment group. Key groups include those sacrificed 10 days post-inoculation. FIG. 6b shows IBA1 FL intensity according to drug treatment. All animals were sacrificed 3 days post-virus inoculation. Results reported as mean FL intensity+/−S.E.M. with *p<0.05. N=7-17 sections per group. FIG. 6c shows IBA1 FL intensity according to drug treatment. All animals were sacrificed 6 days post-virus inoculation. N=10-16 sections per group. FIG. 6d shows IBA1 FL intensity according to positive or negative COVID-19 status and drug treatment. All animals were sacrificed 10 days post-virus inoculation. Results reported as mean FL intensity+/−S.E.M. with *p<0.05 and **p<0.01. N=7-16 sections per group. FIG. 6e shows IBA1 FL intensity nonlinear fit based on time post-inoculation for each COVID-19 positive treatment group (saline, memantine, or ifenprodil). Dotted lines represent 95% CI. The graph's upper right-hand corner lists the slope+/−95% CI, R (correlation coefficient), F statistics, and p-value. N=7-17 sections per group.

### DESCRIPTION

The following description of particular aspect(s) is merely exemplary in nature and is in no way intended to limit the scope of the invention, its application, or uses, which may, of course, vary. This disclosure is provided with relation to the non-limiting definitions and terminology included herein. These definitions and terminology are not designed to function as a limitation on the scope or practice of the disclosure but are presented for illustrative and descriptive purposes only. While the compositions may be described using specific materials in a particular order, it is appreciated that the described materials or order may be interchangeable such that the description effectively includes multiple parts or steps arranged in many ways as is readily appreciated by one of skill in the art.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

The present disclosure concerns the administration or application of one or more N-methyl-D-aspartic acid (NMDA) receptor (NMDAR) antagonists to a subject prophylactically and/or following a viral infection. In some aspects, the administration or application of at least one NMDAR antagonist will prophylactically prevent or reduce the onset and/or severity of neurological side effects and/or neurological damage caused by a viral infection. In some aspects, the administration or application of one or more NMDAR antagonists will treat neuropathologies caused by a viral infection.

In some aspects, the present disclosure concerns methods for preventing and/or treating against adverse neurological effects caused by a viral infection within a subject. In some aspects, the methods can include administration or application of one or NMDAR antagonists to a subject prophylactically and/or following a viral infection. In some aspects, the NMDAR antagonists includes memantine. In some aspects, an NMDAR antagonist may include memantine, ketamine, ifenprodil, donepezil, amantadine, atomoxetine, agmatine, dextrallorphan, dextromethorphan, dextrophan, dizocilpine, neramexane, remacemide, eliprodil, selfotel, phencyclidine or combinations thereof. In some aspects, the NMDAR antagonist is also a nicotinic acetylcholine receptor (nAChR) antagonist, such as memantine, or is administered or applied in combination with a nAChR antagonist, anti-nicotinic or anti-cholinergic. In some aspects, the NMDAR antagonist may be administered with bupropion, dextromethorphan, doxacurium, hexamethonium, mecamylamine, and/or tubocurarine. In some aspects, an NMDAR antagonist may be administered or applied in combination with an anti-viral therapy, such as monoclonal antibodies or active fragments thereof directed against one or more epitopes on the virus, such as bamlanivimab, cilgavimab, sotrovimab, tixagevimab, tocilizumab, or combinations thereof. In some aspects, the anti-viral therapy may include a broad spectrum anti-viral, such as molnupiravir, nirmatrelvir, remdesivir, ritonavir, or combinations thereof. In some aspects, an NMDAR antagonist may be administered or applied in combination with an AMPA (α-amino-3-hydroxy-5-methyl-4-isoxazolepropionic acid) receptor antagonist, such as perampanel. In some aspects, an NMDAR antagonist may be administered or applied in combination with a systemic steroid, such as a corticosteroid, including dexamethasone, hydrocortisone, methylprednisolone, prednisone, or combinations thereof.

In some aspects, the administration and/or application of an NMDAR antagonist is performed as part of a combination therapy with one or more other agents or compounds. It will be appreciated that such can be achieved through formulating all the compounds into one composition, through administration/application of the compounds simultaneously, sequentially, or individually over separate dosing regimens.

In some aspects, the methods of the present disclosure concern administration or application of an NMDAR antagonist such as memantine to a cell of a subject, such as a cell in vitro, in vivo, or ex vivo. In some aspects, the methods of the present disclosure concern administration of an NMDAR antagonist, such as memantine, to a subject, such as a mammal, including a human. In some aspects, the methods may include administration or application of an NMDAR antagonist, such as memantine, to a human subject that has been exposed to or is infected with a virus. As demonstrated in the examples herein, NMDAR antagonists improve survival of subjects exposed to a lethal viral challenge. In some aspects, prophylactic or preventative treatment with an NMDAR antagonist, such as memantine, improves survival of the subject when later encountering a viral infection. It is an aspect of the present disclosure that the administration or application of an NMDAR antagonist, such as memantine, will prevent or treat the neuropathology, neurodysregulation, or neurotoxicity that occurs as a result of a viral infection. In some aspects, a viral infection triggers an inflammatory response that can result, at least in part, in a significant release of cytokines (often referred to as a “cytokine storm”) from the immune system which can in turn overwhelm one or more types of cells or cell processes within the subject. In some aspects, the cytokines overwhelm or overexcite extracellular receptors expressed in particular tissues or on particular cell types. In some further aspects, a viral infection may cause glutamate excitotoxicity which can lead to neuronal damage. In some aspects, the present disclosure has identified that an immune response and/or glutamate excitotoxicity in response to a virus can be prevented, reduced and/or treated, at least in part, through the administration or application of NMDAR antagonists, such as memantine. In some aspects, the treatment is prophylactic. In some aspects, the treatment is following a positive viral infection and/or the onset of symptoms. In some aspects, the treatment is following the onset of neuropathological symptoms.

In some further aspects, the present disclosure concerns administration or application of an NMDAR antagonist to a human. In some aspects, the human is vulnerable to a viral infection. Such vulnerable subsets may include elderly people, immunocompromised people, people with a pre-existing condition such as cardiac issues, people with diabetic issues, people who are obese, people with hematological issues, people with immune system issues, people with auto-immune disorders (e.g. rheumatoid arthritis, systemic lupus erythematosus, Grave's disease, type I diabetes, celiac disease, multiple sclerosis), people with pre-existing hypertension, pregnant patients or patients trying to become pregnant, the presence of a tumor, people undergoing chemotherapy, people undergoing radiation therapy, people with nephrological issues, people with chronic kidney disease, patients receiving hemodialysis or peritoneal dialysis, people with hepatic issues, people with psychiatric illness related to immune function, people with multiple issues/ailments or similar. In some aspects, the subject may have an underlying neurological disorder such as Alzheimer's disease, Parkinson's disease, Huntington's disease, amyotrophic lateral sclerosis, multiple sclerosis, and/or prion disease. It is an aspect of the present disclosure that the prophylactic treatment can reduce the onset or severity of neuropathologies in the instance where the subject contracts a viral infection. The prophylactic treatment of vulnerable people can provide a benefit to both the individual and to those around the individual, particularly where the prophylactic treatment allows the subject's immune responses to more effectively combat the infection.

In some aspects, one or more NMDAR antagonists, such as memantine, can be administered to a human through any known route or combination thereof. In some aspects, an NMDAR antagonist can be administered orally, intravenously, by infusion, transdermally, sublingually, intramuscularly, by inhalation, rectally, vaginally, subcutaneously, ophthalmically, buccally, nasally, enterally, topically, intrathecally, intracerebrally, or combinations thereof. It will be appreciated that other more invasive routes such as intraperitoneal administration can be applied as well, though largely in non-human subjects.

In some aspects, the NMDAR antagonists can be administered with a vehicle, an excipient, and/or a pharmaceutically acceptable carrier. For example, for transdermal administration, the NMDAR may be administered with a composition that assists it crossing the dermal barriers, such as nicotine. In aspects such as where the NMDAR antagonists are directly entering the subject's circulatory system, vehicles such as saline may be utilized. Such additives are understood in the art (see, e.g., Remington: The Science and Practice of Pharmacy, 23^{rd} Edition, A. Adejare ed., 2020).

In some aspects, the methods of the present disclosure utilize one or more NMDAR antagonists, such as memantine, for prophylaxis and/or the treatment of neurological damage or side effects from infection with one or more viruses. As identified in the Examples, herein, the application of an NMDAR antagonist is effective in improving survival against SARS-CoV-2 coronavirus. As has been reported, some SARS-CoV-2 coronavirus infections result in neurological damage. By targeting the NMDAR pharmaceutically, the virus is not able to over-stimulate the receptor, either directly, through triggering a cytokine storm, or creating other conditions leading to excitotoxicity and the damage is averted. It is similarly understood that inflammation caused by peripheral (non-neurological) viruses can result in neurological anomalies or neuropathological manifestations, including acute necrotizing hemorrhagic encephalopathy, encephalitis, Guillain-Barré syndrome, meningitis, audio-visual disabilities, anxiety, acute-flaccid paralysis, inflammatory-demyelinating polyneuropathy, and the like. Accordingly, through the application or administration of an NMDAR antagonist, such as memantine, it is possible to prophylactically or effectively treat against the onset or degradation into these neuropathologies. In some aspects, the methods of the present disclosure concern the prevention or treatment against one or more neuropathologies from a virus. In some aspects, the virus is a peripheral or non-neurotropic virus. In some aspects, the virus is measles virus, a coronavirus, an enterovirus, a adenovirus, an arbovirus, an Arenavirdae, a Bornavirdae, a Flavivirdae (e.g. Chikunga virus, Dengue virus, Hepatitis C, Japanese encephalitis B virus, West Nile virus, St. Louis encephalitis, Zika virus), a Hepadnavirdae (Hepatitis B), a Herpesvirdae (e.g., Herpes simplex type I/II, varicella zoster virus, cytomegalovirus, Epstein-Barr virus, human herpesvirus 6/7, Kaposi's sarcoma virus), human immunodeficiency virus, human T-lymphotropic virus type I, a paramyxovirdae (e.g. Nipah virus), a Picornavirdae (e.g. Hepatitis A), a Rhabdovirdae (e.g. Rabies virus), a Togavirdae, and/or an influenza virus. In certain aspects, the virus is a coronavirus, such as SARS-CoV-1 (or SARS-CoV), MERS-CoV, beta-coronavirus, human coronavirus OC43, and/or SARS-CoV-2. In some aspects, the virus causes coronavirus disease 2019 (COVID-19), such as through one or more variants of SARS-CoV-2, such as omicron, alpha, beta, epsilon, eta, iota, kappa, zeta, and mu, including lineages of B.1.1.7, B.1.1.529, BA.2, BA.1, BA.1.1, B.1.351, P.1, B.1.617.2, C.37, B.1.621, B.1.621.1, B.1.429, B.1.427, CAL.20C, P.2, B.1.525, P.3, B.1.526, B.1.617.1, B.1.617.3, BA.2.86, XBB.1.9.1, XBB.1.9.2, XBB.2.3, XBB.1.16, XBB.1.5, CH.1.1, P.1, and descending lineages thereof (see, e.g., cov-lineages.org and/or covariants.org)

In some aspects, the present disclosure concerns the prevention of a neuropathology in a human subject through the administration of memantine. Memantine may be administered either alone or in combination with an additional NMDAR and/or anti-nicotinic agent and/or an anti-viral agent and/or systemic steroid. In some aspects, memantine is administered to an elderly subject and/or a subject susceptible to viral infection. In some aspects, memantine is administered in general as a preventative or prophylactic treatment. In other aspects, memantine is administered following the positive identification of a viral infection but prior to the onset of symptoms. In some aspects, memantine is administered following a virus exposure or suspected virus exposure. In some aspects, memantine is administered to prevent or lessen neuropathologic manifestations from a viral infection, such as a coronavirus infection, such as infection by SARS-CoV-2 or its variants. In some aspects, administration may continue following a viral infection or a positive test for a viral infection.

In some aspects, the present disclosure concerns the treatment of a neuropathology in a human subject through the administration of memantine. Memantine may be administered either alone or in combination with an additional NMDAR and/or anti-nicotinic agents and/or an anti-viral agents and/or systemic steroid. In some aspects, memantine is administered to an elderly subject and/or a subject susceptible to viral infection. In some aspects, memantine is administered in general following the diagnosis of a viral infection in the subject. In some aspects, memantine is administered following the positive diagnosis of a viral infection but prior to the onset of symptoms. In some aspects, memantine is administered following the onset of one or more neuropathologies in a subject caused by a viral infection or suffering therefrom. In some aspects, memantine is administered following a virus exposure or suspected virus exposure. In some aspects, memantine is administered to treat or reduce neuropathologic manifestations from a viral infection, such as a coronavirus infection, such as infection by SARS-CoV-2 or its variants.

The working examples herein examine if exposure to SARS-CoV-2 triggers neuroinflammatory processes in the respiratory centers of the brain, contributing to the neurological symptoms associated with the illness. Animals exposed to COVID-19 and pre-treated with NMDA receptor antagonists were observed over a course of time for assessment of health and survival outcomes. Then, markers for neuropathology were assessed such as morphological changes, neuronal death and demyelination. Neuroinflammatory markers, namely reactive astrocytes and microglia, were measured in exposed and unexposed pons and medulla tissue from murine subjects to test if inflammatory processes are occurring due to COVID-19.

It has been observed that microglia and astrocyte activation is associated with excitotoxicity^{35,36}, or the overactivation of NMDA receptors causing cell damage and contributing to pro-inflammatory environments²⁰. This is often induced by excessive glutamate, which binds to NMDA receptors to produce excitatory events³⁴. Specifically, the NMDA receptor subunit NR2B binds glutamate and is therefore thought to be most closely associated with glutamate-induced excitotoxic effects^{32,34}. Blocking NMDA receptors with therapeutic levels of memantine, an NMDA receptor antagonist, has demonstrated neuroprotective effects in models simulating hypoxia²⁸ and pro-inflammatory conditions²⁹. In these environments, memantine protected samples from cell damage and demonstrated anti-inflammatory properties by inhibiting microglial activation^{28,29}. Moreover, memantine has been shown to alleviate neuropathology in a mouse model for neuroinvasive human respiratory virus, highlighting its potential efficacy in viral illnesses³⁰. Memantine is a moderate-affinity, non-competitive NMDA antagonist that is known to also block serotonergic and acetylcholine receptors at therapeutic levels³¹. Ifenprodil is another NMDA antagonist that is more specific to the NR2B subunit of the NMDA receptor³².

The results herein found that memantine treatment significantly improved survival in animals positive for COVID-19. Furthermore, both memantine and ifenprodil treatment appear to reduce observable signs of disease (i.e., decreased body weight).

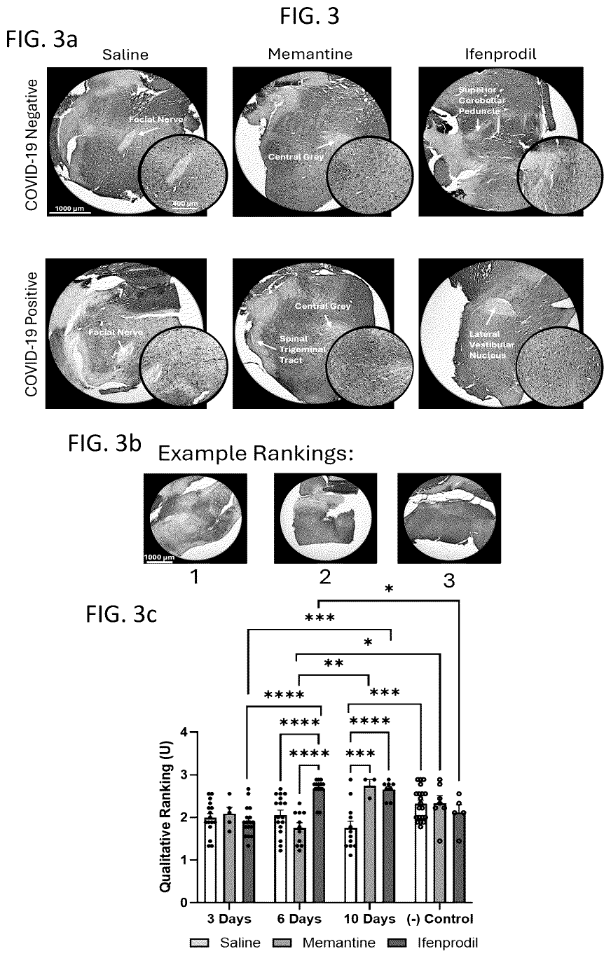

In the presence of COVID-19 infection, brain morphology changes have been reported in the cerebral cortex of humans^{38,39}. To explore possible morphologic alterations in the pons and medulla in the context of COVID-19, each treatment group was stained with cresyl violet and imaged. This allows for visualization of anatomic features, making it a useful method to observe morphological changes due to COVID-19 infection and pre-treatment with a NMDA receptor antagonist. It was found that over time, COVID-19 infection may result in dampened morphology in respiratory regions of the brain compared to tissue not exposed to the virus (FIG. 3). This observation may be due to a surge in cell death caused by SARS-CoV-2 among neurons, glial cells, or both. It is known that SARS-CoV-2 preferentially infects cortical astrocytes over cortical neurons and microglia; however, there is no increase in astrocytic cell death as a result⁴¹. Therefore, it has been hypothesized that astrocyte infection with SARS-CoV-2 indirectly causes neuronal death^{38,41}. To evaluate neuronal death in the respiratory centers of the brain, FJC staining was completed and evaluated for each treatment group. FJC detects degenerating neuronal cell bodies regardless of mechanism by which cell death occurs⁴². The obtained results show that exposure to SARS-CoV-2 or pre-treatment with NMDA receptor antagonists did not have an impact on neuronal death in the pons or medulla. However, there is evidence of an overall neuronal cell reduction in response to COVID-19 in human cortical tissue and organoids^{41,43}.

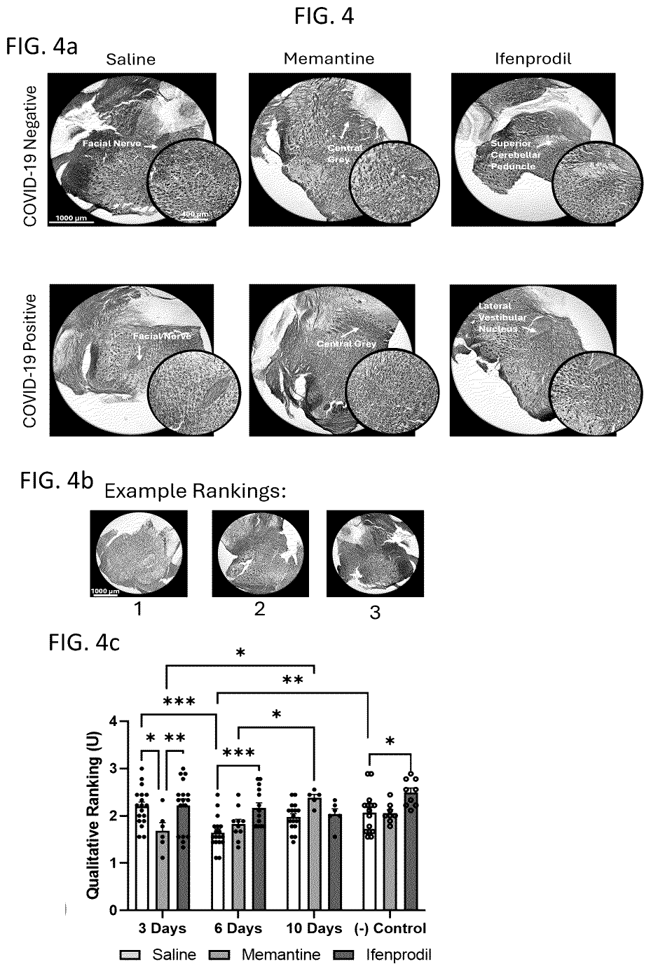

Under normal conditions, astrocytes are important for maintaining hemostasis in the CNS²³. This includes assisting in functions important for myelination¹⁷. As astrocytes are invaded by SARS-CoV-2 inducing a proinflammatory response, disruptions in myelination can occur and have been reported^{23,27,44}. To visualize demyelination in the pons and medulla, sections from each treatment group were stained with Black-Gold II Myelin. Infection with COVID-19 shows signs of demyelination at 6 days post-inoculation compared to the group not exposed to the virus, though by day 10 levels seem to return to baseline (FIG. 4). Loss of myelinated axons in mice following COVID-19 infection has been reported in subcortical white matter in murine subjects as early as 7 days post-infection and persisted for at least 7 weeks²⁰.

Overall effects of NMDA receptor pre-treatment and time were found in both morphology (FIG. 3C) and myelination (FIG. 4C). Considerable changes in morphology induced by COVID-19 over time post-inoculation were observed. Pre-treatment with either memantine or ifenprodil both resulted in morphology rankings similar to the negative control groups by day 10 post-inoculation, signifying that NMDA receptor antagonism may ameliorate COVID-19-induced deficits in morphology. Furthermore, cresyl violet staining ranks generally increased with COVID-19 infection progression in NMDA pre-treated groups, further highlighting that excitotoxicity is a potential source of neuropathology in COVID-19. Myelination rankings followed a different pattern, where pre-treatment with ifenprodil was the only group to seemingly show no differences with COVID-19 inoculation or time course. Memantine treated groups showed signs of demyelination that followed the same trends as the saline treated positive control. It should also be noted that anti-NMDA receptor antibodies have been associated with acute demyelinating encephalitis (ADEM), a neurological complication observed in some COVID-19 patients⁴⁵⁻⁴⁷. In such cases, antagonizing NMDA receptors may exacerbate signs of neuronal injury. Nonetheless, these findings suggest that NMDA receptor antagonism may improve morphological and myelination deficits related to COVID-19.

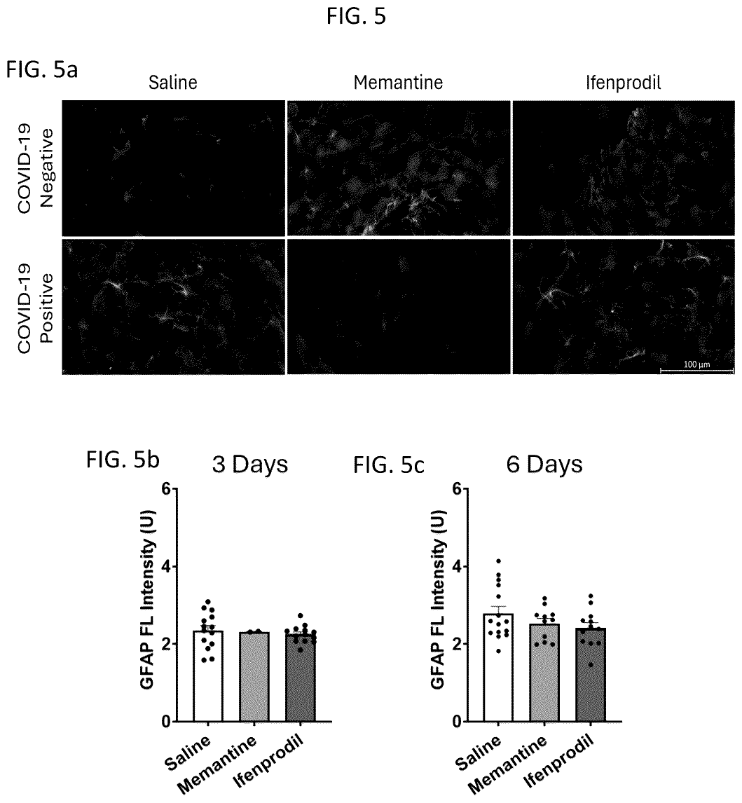

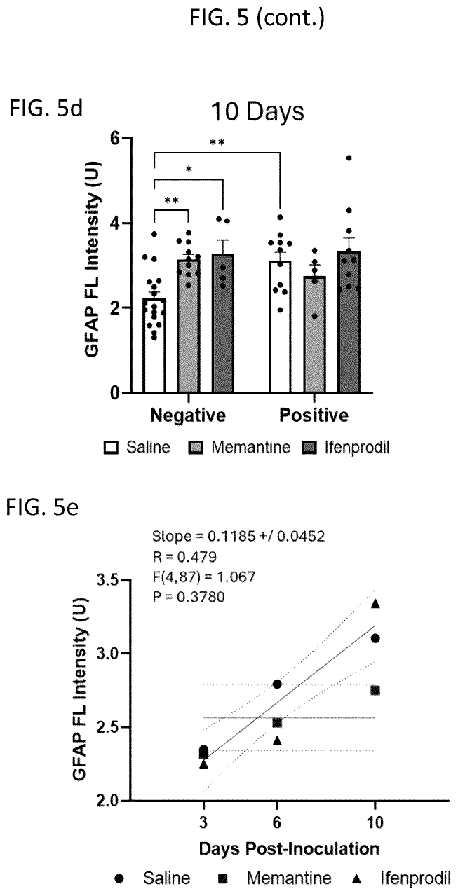

Astrocytes are infected by SARS-CoV-2 preferentially over neurons and microglia^{38,41}. Astrocytes serve as resident glia cells that respond to trauma, inflammation, and infection^{23,48}. There are many ways astrocytes react to pathogen invasion, and the intensity and form of the response heavily depends on the environment^{24,48}. However, there are commonalities among astrocyte reactivity, such as up-regulation of GFAP and increase in cell size^{23,24,49}. IF staining for GFAP can inform on astrocyte reactivity, which when amplified corresponds to a measurable increase in mean FL intensity. Importantly, GFAP staining only indicates the presence of reactive astrocytes, not the underlying function of reactivity. GFAP FL intensity was significantly increased in pons and medulla tissue infected with COVID-19 for 10 days when compared to tissue not exposed (FIG. 5). This suggests that exposure to COVID-19 triggers an immune response within the infected tissue, increasing astrocyte reactivity. Under normal physiologic conditions, astrocytes play a vital role in maintaining extracellular glutamate levels⁵⁰. It is known that in neuroinflammatory environments, astrocytes are unable to maintain homeostatic levels of glutamate, causing excitotoxicity^{51,52}. As set forth herein, pre-treatment with the NMDA receptor antagonist memantine, which indicates prevention of excess glutamate binding to NMDA receptors, may result in the limitation of reactive astrocyte activation in response to COVID-19 (FIG. 5), therefore preventing excitotoxic pathology. However, it is important to note that the groups not inoculated with SARS-CoV-2 but still pre-treated with a NMDA receptor antagonist had a mean GFAP FL intensity that matched that of the control group infected with the virus for 10 days. This establishes that pre-treating healthy tissue with NMDA receptor antagonists may have unknown consequences. Other NMDA receptor antagonists, such as ketamine, are known to cause pathologic morphological changes when administered in moderate to high doses in rats^{53,54}.

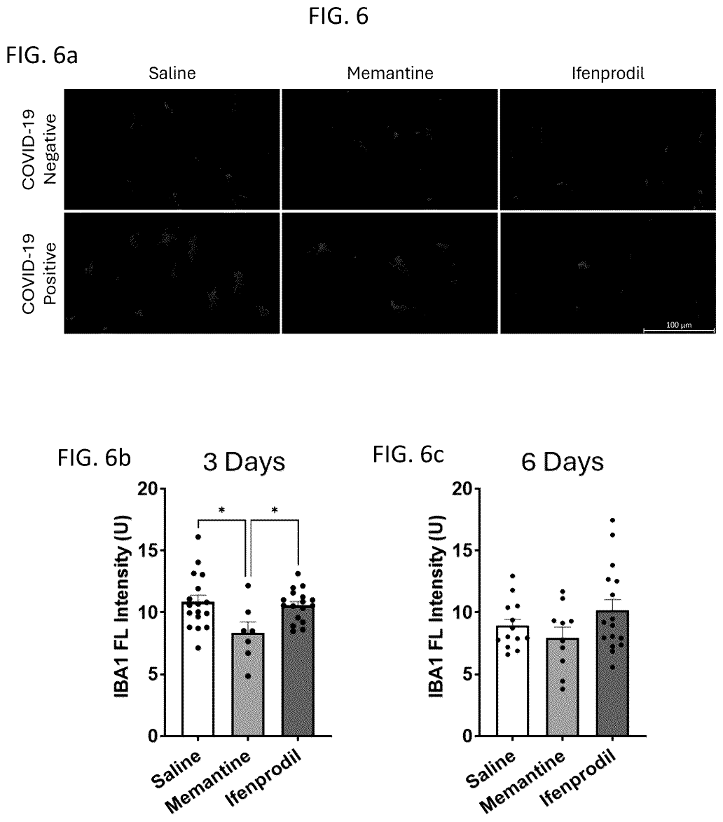

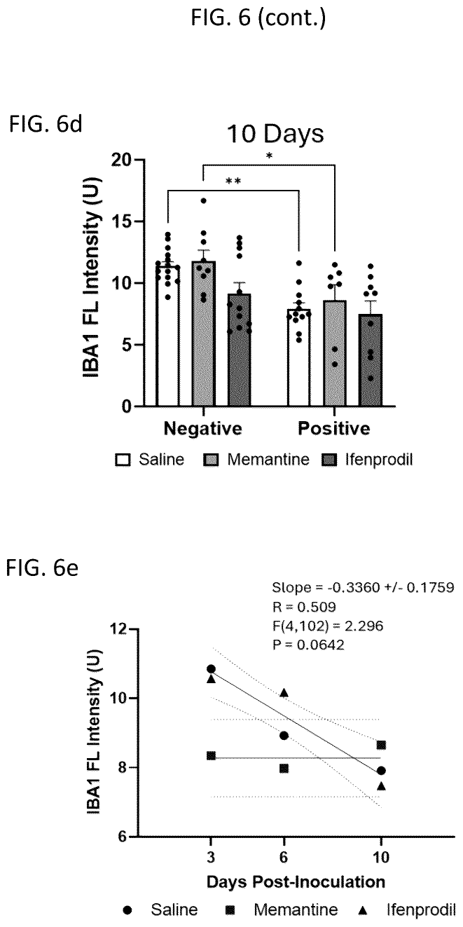

Reactive astrocytes are not the only cells implicated in COVID-19. Neuroinflammation and SARS-CoV-2 infection has been linked to proliferation of microglia^{43,55}. Microglia are known as the central phagocytes inhabiting the CNS⁵⁶. They are important for maintaining neural homeostasis and phagocytose debris in response to injury or infection⁵⁷. They have different functional phenotypes, allowing them to change morphologically based on their activation status and environment⁵⁷. IBA1 is a protein expressed in microglia and peripheral macrophages⁵⁸. IF staining for IBA1 informs on microglial presence but cannot distinguish between activated or non-activated phenotypes⁵⁸. Therefore, heightened IBA1 FL intensity would suggest morphological growth in cell size or increase in microglial numbers, not activation. Furthermore, there is evidence of certain conditions causing a decrease of IBA1⁵⁸; therefore, IF staining for IBA1 cannot distinguish between loss of IBA1 expression and decline in total microglia cells. Interestingly, IBA1 FL intensity levels have the opposite response to progression of COVID-19 infection than GFAP FL intensity levels. IBA1 mean FL intensity in the pons and medulla was significantly decreased in tissue infected with COVID-19 for 10 days when compared to tissue not exposed (FIG. 6). This conflicts with other reports of increased microglial signal after infection with SARS-CoV-2^{41,43}. Instead, the results herein establish a pattern with GFAP increasing in mean FL intensity and IBA1 decreasing in mean FL intensity with progression of COVID-19 infection (FIGS. 5&6). This could be due to the crosstalk between astrocytes and microglia, activating a compensatory mechanism in response to impaired microglial activity⁵⁹. Previous studies have shown that infections can cause microglial pryoptosis, a type of inflammatory programmed cell death⁶⁰. Specifically, herpes simplex virus type 1 (HSV-1) and human immunodeficiency virus-1 (HIV-1) cause pryoptosis in microglia through separate mechanisms, contributing to the neuropathology observed in each of these illnesses^{61,62}. It has been previously demonstrated that astrocytes are activated due to the depletion of microglia and will phagocytose microglial fragments^{63,64}. If microglia are impaired due to SARS-CoV-2 infection, astrocytes would become activated and compensate for microglial damage. This process is unique to astrocytes and is one explanation as to why there would be an increasing reactive astrocyte signal alongside a decreasing microglia signal. It has also been observed that, under neuroinflammatory conditions, astrocytes themselves can suppress microglial activation through acute-phase protein production⁶⁵.

The present results show similar GFAP and IBA1 FL intensity trends between the control and ifenprodil pre-treated groups, suggesting that ifenprodil did not have a preventative effect in COVID-19 induced neuroinflammation in the respiratory centers of the brain (FIGS. 5&6). However, it is observed that mean IBA1 FL intensity in animals sacrificed 3 days post-virus inoculation were slightly lower in the memantine-treated group than the control group (FIG. 6). Additionally, when mice were pre-treated with memantine and sacrificed 10 days post-virus inoculation, IBA1 mean FL intensity was slightly lower than the memantine treated control group sacrificed 10 days post-virus inoculation (FIG. 6). Memantine has a previously defined anti-inflammatory mechanism in which it increases astrocyte functionality to promote neuronal growth while also inhibiting activation of microglia in the midbrain.²⁹ This suggests that microglial activation may be inhibited by memantine causing a lower mean IBA1 FL intensity overall, demonstrating that memantine may play a neuroprotective role in pons and medulla tissue exposed to COVID-19. Moreover, the memantine pre-treated group showed no change in mean IBA1 or GFAP FL intensity with progression of COVID-19 infection while the control and ifenprodil pre-treated groups both were significantly changed (FIGS. 5&6). This further illustrates the possible utility memantine has as a preventative agent for neuroinflammation. However, this result must be considered carefully, as our analyses also did not identify the curves representing each treatment over time were to be significantly different from one another. If there had been a longer exposure time allowing the virus to further exacerbate pro-inflammatory processes, a larger change in memantine-treated animals may have been observed. The lack of significance and conflicting results warrant future studies that include longer exposure times to SARS-CoV-2 to clarify the impact memantine has on neuroinflammatory processes in the respiratory centers of the brain.

As set forth herein, there is an increase of reactive astrocyte signal in response to COVID-19 exposure in the pons and medulla. This finding suggests the presence of neuroinflammation, which is consistent with the original hypothesis that COVID-19 exposure triggers neuroinflammatory processes in the respiratory centers of the brain. As also shown herein, when mice are pre-treated with memantine, there is a protective effect against COVID-19 induced neurological symptoms as well as neuroinflammation.

### EXAMPLES

### Animal Studies

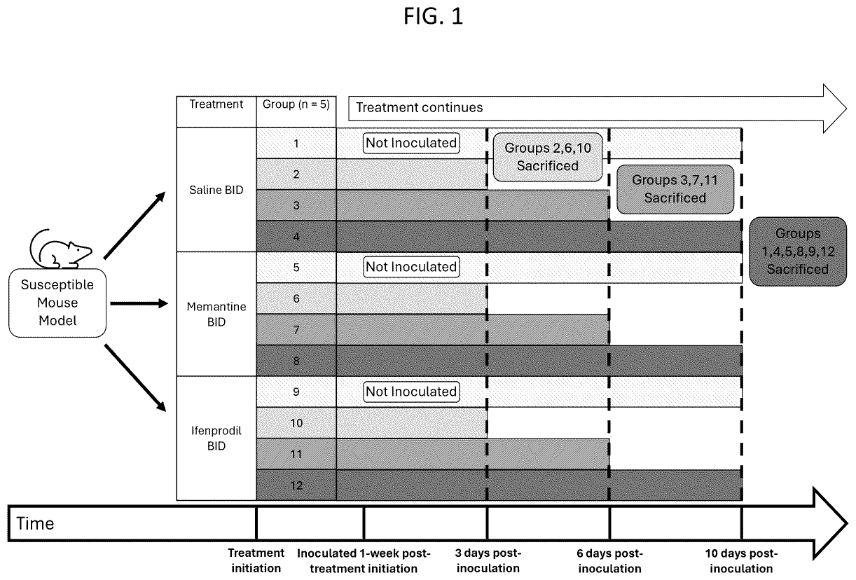

Animals were allocated into 12 groups, with n=5 in each group. Groups 1-4 were pretreated with saline, with groups 2-4 receiving intranasal inoculation of SARS-CoV-2, Beta variant, 1-week post-treatment initiation. Groups 5-8 were pretreated with memantine (10 mg/kg I.P. twice daily), and groups 6-8 were inoculated with the same virus 1 week later. Groups 9-12 were pretreated with ifenprodil (10 mg/kg I.P. twice daily), and groups 10-12 were inoculated with the same virus 1 week later. Therefore, groups 1, 5, and 9 were not inoculated with COVID-19 allowing for controlled comparisons within each treatment. To understand how disease progression affects the neuropathology examined in this study, groups were sacrificed at different time points. Groups 2, 6, and 10 were sacrificed 3 days after virus inoculation. Groups 3, 7, and 11 were sacrificed on day 6 post-inoculation and the remaining were sacrificed on day 10. Experimental design, including COVID-19 inoculation status, treatment, time course for each group is shown in FIG. 1. FIG. 1 sets forth information concerning the morbidity with decline in body weight utilized as the determining factor, with all mice receiving saline/vehicle failing to survive past 8 days after the viral inoculation. Mice receiving memantine showed good survival, with ifenprodil also showing some protective effect.

### Drugs and Administration

### SARS-CoV-2 Inoculation

All experiments with SARS-CoV-2 were performed in enhanced biosafety level 3 (BSL3) containment laboratories at the University of Louisville, which are approved for such use by the Centers for Disease Control and Prevention and by the US Department of Agriculture. Mice were exposed to SARS-CoV-2 via intranasal inoculation. Following inoculation with SARS-CoV-2, animals were monitored throughout the experiment for observable health effects, including change in body weight and survival outcomes.

### NMDA Receptor Antagonists

Mice were injected twice daily (intraperitoneal (I.P.)) with either saline, memantine (10 mg/kg), or ifenprodil (10 mg/kg) one week prior to inoculation. Doses were determined by allometric scaling. Injections began one week prior to SARS-CoV-2 exposure to reach steady-state plasma concentrations of the compounds and continued twice daily (BID) until the assigned tissue collection day (FIG. 1).

### Tissue Collection, Fixation, and Slicing

Mice were anesthetized and the brains were removed and placed in the same fixative for 48-72 hours at room temperature, followed by cryoprotection in 30% sucrose for 3 days at 4° C. before storage at −80° C. The right hemisphere of each fixed brain was sectioned coronally through the entire structure using a cryostat with a fine section set to 45 nm. Slices were collected in a 12-well plate filled with 0.1% sodium azide (1 g sodium azide mixed into 100 mL 10×PBS and 900 mL of distilled water in laboratory fume hood; ThermoFisher Scientific, CAS 26628-22-8) and stored in 4° C. fridge. Slices were mounted onto Superfrost Plus slides (ThermoFisher Scientific) in a distilled water bath prior to staining. Slides were air dried overnight to allow for tissue adhesion.

### Cresyl Violet Staining

To assess general morphology, cresyl violet staining was utilized for each group. The staining procedure was completed in a laboratory fume hood to limit exposure to hazardous reagents. 1.25 g of cresyl violet acetate (Acrōs Organics, Geel, Belgium) was dissolved in 250 mL of warm distilled water. Glacial acetic acid (30 nM; ThermoFisher Scientific, CAS 64-19-7) was added to the mixture, which was then cooled and filtered. Slides were immersed in a descending alcohol series to allow for rehydration prior to staining: 100% ethanol, followed by 95%, 70%, then distilled water to rinse. Sections were stained in cresyl violet solution for 3 minutes. Slides were rinsed with distilled water and dehydrated in an ascending alcohol series (i.e. reverse order of ethanol dehydration). Finally, slides were cleared in CitriSolv (Decon Labs, King of Prussia, PA, USA. CAS 5989-27-5) for 5 minutes, coverslipped with Vectamount mounting medium (Vector, Cat. H-500, Newark, CA, USA), and imaged using bright field microscopy.

### Black-Gold II Myelin and Fluoro-Jade C Staining

The current study aimed to visualize protential demyelination and neuronal death among mice inoculated with COVID-19. Sections were stained with Black-Gold II Myelin Stain Reagent with Toluidine Blue O Counter Stain kit (Biosensis, Thebarton, SA, Australia; Cat. TR-100-BG) according to manufacturer instructions. Slides were imaged using bright field microscopy to estimate myelination in each group. To visualize cell death, a set of adjacent sections from each group were stained with Fluoro-Jade C Stain kit (Biosensis, Thebarton, SA, Australia; Cat. TR-100-FJT) according to manufacturer instructions. The staining procedure was completed in a laboratory fume hood to limit exposure to hazardous reagents. The slides were imaged using the appropriate fluorescent microscopy settings on the Zeiss Slide Scanner Axioscan Z7.

### Immunofluorescence

Another set of adjacent sections from each group were processed for GFAP and IBA-1 immunofluorescence (IF) to visuaflize astrocytes and microglia, respectively. Sections undergoing GFAP IF were incubated in Rabbit anti-GFAP primary antibody, DAKO #Z033401-2 (1:1000, Agilent, Santa Clara, CA, USA) overnight, followed by a 2-hour incubation in Goat anti-Rabbit IgG (H+L) Cross-Adsorbed Secondary Antibody, Alexa Flour™ 488, #A11008 (1:500, Invitrogen, Waltham, MA, USA). Similarly, sections undergoing IBA-1 IF were incubated overnight in goat anti-IBA-1, Wako #019-19741 primary antibody (1:2500, FUIJFILM, Minato City, Tokyo, Japan) then for 1 hour in Goat anti-Rabbit IgG (H+L) Cross-Adsorbed Secondary Antibody, Alexa Fluor™ Plus 647, #A32733 (1:500, Invitrogen). Imaging was completed for each IF stain using the appropriate fluorescent microscopy settings on the Zeiss Slide Scanner Axioscan Z7.

### Analysis

General morphological stains, such as cresyl violet and Black-Gold II Myelin, were utilized as a qualitative measure of neuropathology visualized in the respiratory centers of the brain. A ranking system assessing the visibility of the stain was developed to analyze these images. Nine individuals blinded to the treatment groups ranked stains with a value corresponding to visual assessment of morphology and myelination, respectively. Average ranks across all nine blinded observers were used to determine visual differences between treatment groups for both stains. In the case of the Fluoro-Jade C and IF stains, the images underwent processing through Zeiss software (ZEN Desk 2.5) which included deconvolution and background subtraction. Then, fluorescent intensity was measured for each image within the region of interest using National Institutes of Health ImageJ software. All statistical analyses were done in GraphPad Prism 10.2.0 (GraphPad Software, La Jolla, CA, USA). Each experimental procedure was analyzed using two-way ANOVAs unless otherwise stated. All ANOVAs were followed by Sidak's multiple comparison tests. Nonlinear regression analyses were completed for Body Weight results to identify differences in weight between treatments over time. Nonlinear regression analyses were also completed for GFAP and IBA1 results to identify differences between treatment in the time course of each experimental measure.

### Results

### Survival and Health Outcomes

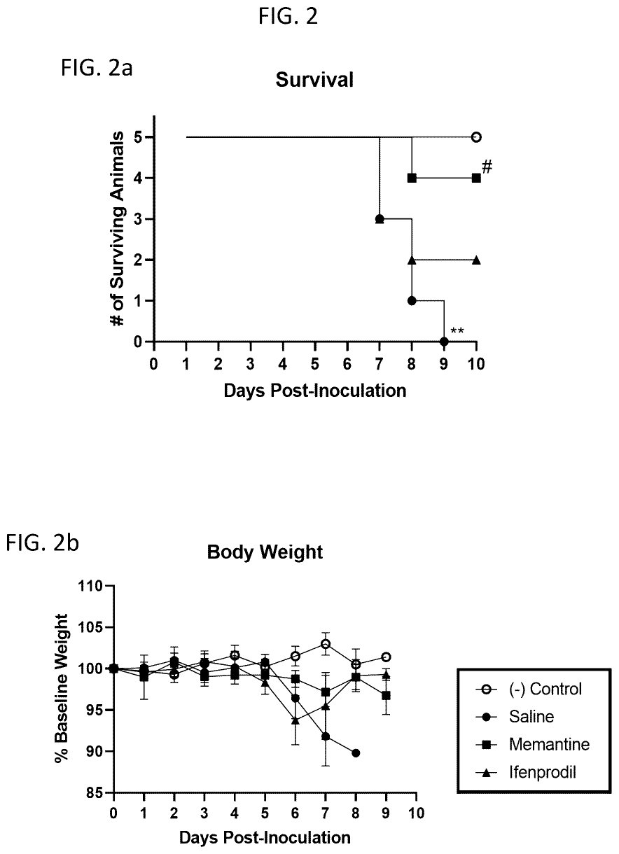

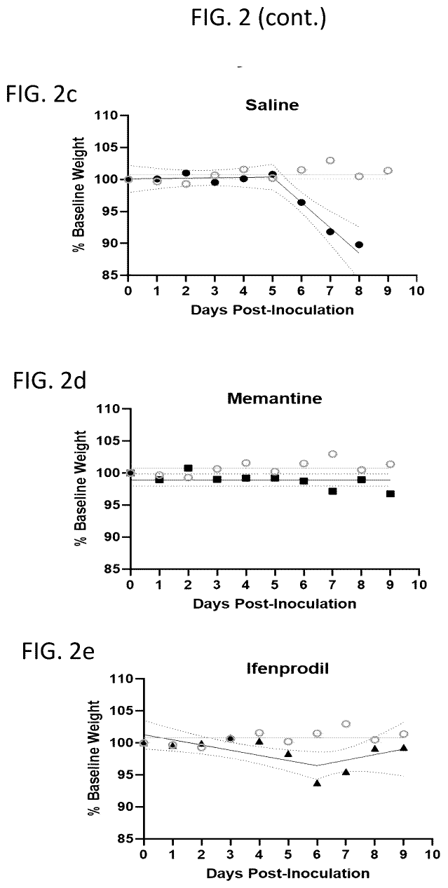

Following inoculation with SARS-CoV-2, animals were monitored throughout the experiment for observable health effects. Survival and body weight, as a measure of disease incidence, are reported for groups 1, 4, 8, and 12 (i.e., the (−) control and the 10 day post-inoculation groups treated with saline, memantine, and ifenprodil, respectively) (FIG. 2). No animals died in the group negative for COVID-19; however, in the saline treated group positive for COVID-19, all animals were either found dead or were euthanized (and tissue collected) due to meeting humane endpoint criteria³⁷. The groups treated with NMDA receptor antagonists had fewer loss of animals, and the remaining animals were sacrificed at the aforementioned 10 day timepoint for analyses. Analysis using a two-way ANOVA of the number of surviving animals per treatment group over time shows that both time and treatment effect survival outcomes (FIG. 2A, Main effect of drug treatment F[3,28]=5.288, p=0.0053; main effect of time F[9,27]=4.161, p=0.0019; two-way ANOVA). Survival outcomes are significantly worsened in COVID-19 positive animals compared to negative ((−) Control vs Saline, p=0.0086, post-hoc analyses). Pre-treatment with memantine, however, resulted in a significantly improved survival outcome (Saline vs memantine, p=0.0447, post-hoc analyses).

Body weight is used as a measure of disease (FIG. 2B-E); as COVID-19 progresses, decreased body weight is indicative of worsened disease state. Body weight is reported as percent of baseline weight, measured prior to inoculation. Nonlinear regression analyses of each group are shown separately for saline (FIG. 2C), memantine (FIG. 2D), and ifenprodil (FIG. 2E) compared to the negative control for case of viewing. We found that animals negative for COVID-19 had little to no fluctuation in body weight (FIG. 2B-E, Best fit: Horizontal Line), while saline treated animals positive for COVID-19 had a significant reduction in body weight starting around day 5 post-inoculation (FIG. 2C, Best fit: Segmental Line; X0=5, Slope1=0, Slope2=-3.96). COVID-19 positive animals treated with memantine, similar to the negative control, had little to no fluctuation in body weight (FIG. 2D, Best fit: Horizontal Line). Interestingly, COVID-19 positive animals treated with ifenprodil showed a reduction in body weight starting around day 5, similar to the saline treated group, though body weight returned to normal levels by day 10 post-inoculation (FIG. 2E, Best fit: Segmental Line; X0=6, Slope 1=-0.8076, Slope2=0.8604). We posit that this is due to the death of animals more heavily impacted by COVID-19, as the return to baseline weights occurs after day 8, which is after the last death in this group. Nonetheless, these results clearly outline the health impacts of SARS-CoV2 and show how pre-treatment with a NMDA receptor antagonist may reduce the severity of these outcomes.

### Semi-Qualitative Analysis of Cresyl Violet and Black Gold II Myelin

To visualize morphological changes to neuronal structure, samples of pons and medulla from each group underwent processing with cresyl violet, which stains the Nissl substance in the neurons of the brain a blue/purple color. Upon visual inspection, lighter staining, indicating loss of anatomical organization, is seen overall in the sections infected with the virus for 10 days versus sections not exposed (FIG. 3).

To further examine the images qualitatively, individuals blinded to the treatment groups were asked to rank the images 1-3, with 1 considered to be an image with increased amount of white zones in the morphological stain and 3 as a more typical morphology. The ranks for each image were then averaged and analyzed for each treatment group (FIG. 3), with higher ranks suggesting a more complete morphological anatomy and lower ranks suggesting morphological alterations due to disease. Comparison of ranks for each treatment group across 3, 6, and 10 days post-inoculation, as well as a COVID-19 negative control, was done using a 2-way ANOVA, with Sidak's post-hoc analyses. Drug treatment with memantine or ifenprodil, as well as time post-inoculation, each had an effect on morphological rankings (FIG. 3C, Main effect of drug treatment F[2, 123]=7.359, p=0.0010; main effect of time F[3, 123]=3.798, p=0.0120; interaction F[6, 123], p<0.0001; two-way ANOVA). In COVID-19 positive subjects pre-treated with saline vehicle, the rank given for morphology is significantly lower at 10 days post-inoculation compared to a negative control (Saline: 10 Days vs (−) Control, p=0.0009, post-hoc analyses). In COVID-19 positive subjects pre-treated with memantine, while 6 days post-inoculation is significantly lower than the negative control similar to saline-treated outcomes, we find that by day 10-post inoculation there is no longer a difference in morphological rank (Memantine: 6 days vs (−) control, p=0.0169; 10 days vs (−) control, p=0.5772; 6 days vs 10 days, p=0.0011, post-hoc analyses). In COVID-19 positive subjects pre-treated with ifenprodil, we initially see no difference in morphological rank at 3 days post-inoculation compared to negative control, however we observe an increase in rankings at 6 and 10 days post-inoculation to a level perhaps slightly above the negative control (Ifenprodil: 3 days vs (−) control, p=0.9065; 3 days vs 6 days, p<0.0001; 3 days vs 10 days, p=0.0001; 6 days vs (−) control, p=0.0387, post-hoc analyses).

In subjects inoculated with the negative control virus, no differences were observed with drug treatment. Similarly, no differences were observed across drug treatment at 3 days post-inoculation with SARS-CoV-2. However, at 6 days post-inoculation, we observe that ifenprodil increases morphological rank compared to saline and memantine treatment (6 days: saline vs ifenprodil, p<0.0001; memantine vs ifenprodil, p<0.0001, post-hoc analyses). Similarly, at 10 days post-inoculation, we observe that both memantine and ifenprodil improve the morphological rank compared to saline treatment (10 days: saline vs memantine, p=0.0005; saline vs ifenprodil, p<0.0001, post-hoc analyses). Overall, these results suggest that infection with COVID-19 causes morphological deficits in the pons and medulla over time post-inoculation, while pre-treatment with ifenprodil and memantine may ameliorate this deficit by day 6 or 10 post-inoculation, respectively.

Similar to the cresyl violet stain, Black-Gold II staining was used to visualize any changes in the myelin of the pons and medulla due to exposure to COVID-19 and treatment with a NMDA receptor antagonist. This process directly stained the myelin a maroon color, allowing for clear visualization of differences. A light-blue counter Nissl stain can also be observed in these images (FIG. 4). Again, to further examine the images qualitatively, individuals blinded to the treatment groups were asked to rank the images 1-3, with 1 representing lessened myelination and 3 representing typical or greater myelination. The ranks for each image were averaged and analyzed as before (FIG. 4C). As observed with cresyl violet, we found that drug treatment and time post-inoculation each influenced myelination rankings (FIG. 4C, Main effect of drug treatment F[2,131]=6.370, p=0.0023; main effect of time F[3,131]=4.764, p=0.0035; interaction F[6,131]=3.604, p=0.0024, two-way ANOVA). In COVID-19 positive animals pre-treated with saline vehicle, subjects show signs of demyelination as the myelination ranking is significantly decreased at 6 days post-inoculation compared to both the negative control and 3 days post-inoculation; yet, the myelination ranking is back to negative control levels by day 10 post-inoculation (Saline: 6 days vs (−) control, p=0.0097; 3 days vs 6 days, p=0.0001; 10 days vs (−) control, p=0.9822, post-hoc analyses). Similarly, at 3 days and 6 days post-inoculation, COVID-19 positive mice pre-treated with memantine have decreased myelination rankings (Memantine: 3 days vs 10 days, p=0.0176; 6 days vs 10 days, p=0.0479; 3 days: saline vs memantine, p=0.0139; memantine vs ifenprodil, p=0.0098, post-hoc analyses). However, similar to saline-treated mice, this effect is not observed at 10 days post-inoculation, as we find myelination rankings of memantine pre-treated subjects to be not significantly different at 10 days post-inoculation compared to the negative control (Memantine: 10 days vs (−) control, p=0.5862).

In subjects without SARS-CoV-2, subjects pre-treated with ifenprodil showed an increase in myelination ranking compared to saline ((−) control: saline vs ifenprodil, p=0.0245, post-hoc analyses). Ifenprodil pre-treatment also resulted in an increased myelination ranking at 6 days post-inoculation compared to saline pre-treated animals (6 days: saline vs ifenprodil, p=0.0005, post-hoc analyses), though no significant difference between myelination rankings of ifenprodil pre-treated mice across any time point post-inoculation were observed, regardless of COVID-19 status. By day 10 post-inoculation, we observe no differences between drug treatment groups. Our findings suggest that ifenprodil, but not memantine, may prevent COVID-19 related changes in myelination in the respiratory centers of the brain.

### Fluoro-Jade C Stain

Along with the qualitative data assessing morphology and myelination, fluorescent imaging of neuronal death, microglia, and astrocytes were quantified for a more accurate evaluation of the neuropathology seen in COVID-19 and how it is affected by NMDA receptor antagonism. Firstly, fluoro-jade C (FJC) was utilized to visualize any neuronal death in the pons and medulla. Each section within the areas of interest were analyzed for fluorescent (FL) intensity. No significant differences between mean FL intensity were observed among groups, nor were there any clear patterns suggesting exposure to COVID-19, treatment with a NMDA receptor antagonist, or disease progression affected neuronal death.

### Immunofluorescent Stains

Immunofluorescent (IF) staining processes were used to visualize astrocytes and microglia within the pons and medulla tissue. Glial fibrillary acidic protein (GFAP) is a structural protein specific to reactive astrocytes, while ionized calcium-binding adapter molecules (IBA1) are specific to microglia in the CNS. Therefore, staining these targets using IF processes would allow for characterization of astrocytes and microglia in the areas of interest. In each group, pons and medulla tissue sections were assessed for mean GFAP and IBA1 FL Intensity. The GFAP data was first organized based on COVID-19 status and drug treatment, comparing only groups sacrificed on day 10 post-virus inoculation (FIG. 5D). The analyses show that drug treatment with memantine and ifenprodil influenced mean GFAP FL intensity (main effect of drug treatment F[2,54]=3.921, p=0.0257; two-way ANOVA). Furthermore, the effect of drug treatment on mean GFAP FL intensity was dependent upon a group's COVID-19 status (interaction F[2,54]=4.258, p=0.0192). It is also observed that mean GFAP FL intensity is significantly increased in the positive control group when compared to the negative control group (Saline: negative vs positive, p=0.0046, post-hoc analyses). Interestingly, mean GFAP FL intensity is also increased in comparison to the negative control group in animals negative for COVID-19 but pre-treated with NMDA-receptor antagonists (Negative: saline vs memantine, p=0.0030; saline vs ifenprodil, p=0.0125, post-hoc analyses).

Mean GFAP FL intensity was further analyzed based on time sacrificed post-virus inoculation and drug treatment in groups positive for COVID-19. Using an ordinary one-way ANOVA for analyses at both 3 days (FIG. 5B) and 6 days (FIG. 5C) post-inoculation, we observed no differences between GFAP FL intensity across treatment groups. However, upon plotting the GFAP FL intensity over time (FIG. 5E), we found that disease progression did not have the same effect in subjects pre-treated with memantine as seen in groups pre-treated with saline and ifenprodil. In the memantine group, nonlinear fit analyses showed that increasing time point was not associated with increasing mean GFAP FL intensity (Best Fit: Horizontal Line). However, it cannot be concluded that mean GFAP FL intensity over time is significantly different in the memantine group, as further analysis showed that one curve fits all drug treatments groups plotted over the time course (Best Fit: First-Order Polynomial, R=0.479). Together, our GFAP FL intensity results suggest that pre-treatment with memantine or ifenprodil may increase astrocytic response in naive groups; however, in COVID-19 positive groups, memantine may limit the reactive astrocyte response that is induced by COVID-19.

Similar to GFAP, IBA1 data was first separated by COVID-19 status and drug treatment, only using groups sacrificed on day 10 post-virus inoculation (FIG. 6D). Two-way ANOVA analysis showed that positive or negative COVID-19 status influenced mean IBA1 FL intensity regardless of drug treatment (main effect of COVID status F[1,58]=19.02, p<0.0001, no interaction F[2,58]=0.7979, p=0.4552, two-way ANOVA). Mean IBA1 FL intensity is significantly decreased in the positive control group when compared to the negative control group (Saline: negative vs positive, p=0.0018, post-hoc analyses). Mean IBA1 FL intensity is also significantly decreased in the positive memantine group when compared to the negative memantine group (Memantine: negative vs positive, p=0.0401, post-hoc analyses). At 10 days post-inoculation, no differences were observed between drug treatments in subjects either negative or positive for COVID-19.

Mean IBA1 FL intensity was also further analyzed based on time sacrificed post-virus inoculation and drug treatment in groups positive for COVID-19 using an ordinary one-way ANOVA at 3 and 6 days as above. At 3 days post-inoculation (FIG. 6B), drug treatment was found to have a significant effect on GFAP FL intensity (main effect F[2,38]=4.502, p=0.0176, one-way ANOVA), with memantine specifically having a decreased expression of GFAP compared to saline and ifenprodil treated groups (saline vs memantine, p=0.0180; memantine vs ifenprodil, p=0.0408, post-hoc analyses). At 6 days post-inoculation (FIG. 6C), no differences were observed across drug treatments. Like the effects observed in the GFAP memantine groups, nonlinear fit analysis IBA1 FL intensity over time showed that increasing time point was not associated with decreasing mean IBA1 FL intensity in the group pre-treated with memantine (FIG. 6E, Best Fit: Horizonal Line). However, similar to the GFAP data, it cannot be concluded that mean IBA1 FL intensity over time is significantly different in the memantine group, as further analysis found that there is one curve that fits all drug treatments groups plotted over the time course (Best Fit: First-Order Polynomial, R=0.509). Together, these results suggest that memantine may limit the progression of microglial depletion in the pons and medulla that is induced by COVID-19, though possibly by maintaining microglia at a baseline level that is lower than COVID-19 negative subjects.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary.

Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

### ADDIN EN.REFLIST REFERENCES

1 Umakanthan, S. et al. Origin, transmission, diagnosis and management of coronavirus disease 2019 (COVID-19). Postgrad Med J 96, 753-758 (2020). doi.org:10.1136/postgradmedj-2020-138234
2 Chen, N. et al. Epidemiological and clinical characteristics of 99 cases of 2019 novel coronavirus pneumonia in Wuhan, China: a descriptive study. Lancet 395, 507-513 (2020). doi.org:10.1016/S0140-6736 (20) 30211-7
3 Harapan, B. N. & Yoo, H. J. Neurological symptoms, manifestations, and complications associated with severe acute respiratory syndrome coronavirus 2 (SARS-CoV-2) and coronavirus disease 19 (COVID-19). J Neurol 268, 3059-3071 (2021). doi.org:10.1007/s00415-021-10406-y
4 Shehata, G. A. et al. Neurological Complications of COVID-19: Underlying Mechanisms and Management. Int J Mol Sci 22 (2021). doi.org:10.3390/ijms22084081
5 Mao, L. et al. Neurologic Manifestations of Hospitalized Patients With Coronavirus Disease 2019 in Wuhan, China. JAMA Neurol 77, 683-690 (2020). doi.org:10.1001/jamaneurol.2020.1127
6 Meinhardt, J. et al. Olfactory transmucosal SARS-CoV-2 invasion as a port of central nervous system entry in individuals with COVID-19. Nat Neurosci 24, 168-175 (2021). doi.org:10.1038/s41593-020-00758-5
7 Li, Y. C., Bai, W. Z. & Hashikawa, T. The neuroinvasive potential of SARS-CoV2 may play a role in the respiratory failure of COVID-19 patients. J Med Virol 92, 552-555 (2020). doi.org:10.1002/jmv.25728
8 Song, W. J. et al. Confronting COVID-19-associated cough and the post-COVID syndrome: role of viral neurotropism, neuroinflammation, and neuroimmune responses. Lancet Respir Med 9, 533-544 (2021). doi.org:10.1016/S2213-2600 (21) 00125-9
9 Hoffmann, M. et al. SARS-CoV-2 Cell Entry Depends on ACE2 and TMPRSS2 and Is Blocked by a Clinically Proven Protease Inhibitor. Cell 181, 271-280 e278 (2020). doi.org:10.1016/j.cell.2020.02.052
10 Cantuti-Castelvetri, L. et al. Neuropilin-1 facilitates SARS-CoV-2 cell entry and infectivity. Science 370, 856-860 (2020). doi.org:10.1126/science.abd2985
11 Daly, J. L. et al. Neuropilin-1 is a host factor for SARS-CoV-2 infection. Science 370, 861-865 (2020). doi.org:10.1126/science.abd3072
12 Avdonin, P. P., Rybakova, E. Y., Trufanov, S. K. & Avdonin, P. V. SARS-CoV-2 Receptors and Their Involvement in Cell Infection. Biochem (Mosc) Suppl Ser A Membr Cell Biol 17, 1-11 (2023). doi.org:10.1134/S1990747822060034
13 Jackson, C. B., Farzan, M., Chen, B. & Choe, H. Mechanisms of SARS-CoV-2 entry into cells. Nat Rev Mol Cell Biol 23, 3-20 (2022). doi.org:10.1038/s41580-021-00418-x
14 Ortiz, M. E. et al. Heterogeneous expression of the SARS-Coronavirus-2 receptor ACE2 in the human respiratory tract. EBioMedicine 60, 102976 (2020). doi.org:10.1016/j.ebiom.2020.102976
15 Gao, C. C. et al. Differential transcriptomic landscapes of multiple organs from SARS-CoV-2 early infected rhesus macaques. Protein Cell 13, 920-939 (2022). doi.org:10.1007/s13238-022-00915-5
16 Baughn, L. B. et al. Targeting TMPRSS2 in SARS-CoV-2 Infection. Mayo Clin Proc 95, 1989-1999 (2020). doi.org:10.1016/j.mayocp.2020.06.018
17 Wan, D. et al. Neurological complications and infection mechanism of SARS-CoV-2. Signal Transduct Target Ther 6, 406 (2021). doi.org:10.1038/s41392-021-00818-7
18 Vitale-Cross, L., Szalayova, I., Scoggins, A., Palkovits, M. & Mezey, E. SARS-CoV-2 entry sites are present in all structural elements of the human glossopharyngeal and vagal nerves: clinical implications. bioRxiv (2022). doi.org:10.1101/2021.12.30.474580
19 Ellenberger, H. H. & Feldman, J. L. Brainstem connections of the rostral ventral respiratory group of the rat. Brain Res 513, 35-42 (1990). doi.org:10.1016/0006-8993 (90) 91086-v
20 Fernandez-Castaneda, A. et al. Mild respiratory COVID can cause multi-lineage neural cell and myelin dysregulation. Cell 185, 2452-2468 e2416 (2022). doi.org:10.1016/j.cell.2022.06.008
21 Skaper, S. D., Facci, L., Zusso, M. & Giusti, P. An Inflammation-Centric View of Neurological Disease: Beyond the Neuron. Front Cell Neurosci 12, 72 (2018). doi.org:10.3389/fncel.2018.00072
22 Paolicelli, R. C. et al. Synaptic pruning by microglia is necessary for normal brain development. Science 333, 1456-1458 (2011). doi.org:10.1126/science.1202529
23 Lee, H. G., Lee, J. H., Flausino, L. E. & Quintana, F. J. Neuroinflammation: An astrocyte perspective. Sci Transl Med 15, eadi7828 (2023). doi.org:10.1126/scitranslmed.adi7828
24 Lawrence, J. M., Schardien, K., Wigdahl, B. & Nonnemacher, M. R. Roles of neuropathology-associated reactive astrocytes: a systematic review. Acta Neuropathol Commun 11, 42 (2023). doi.org:10.1186/s40478-023-01526-9
25 Joshi, A. U. et al. Fragmented mitochondria released from microglia trigger A1 astrocytic response and propagate inflammatory neurodegeneration. Nat Neurosci 22, 1635-1648 (2019). doi.org:10.1038/s41593-019-0486-0
26 Liddelow, S. A. et al. Neurotoxic reactive astrocytes are induced by activated microglia. Nature 541, 481-487 (2017). doi.org:10.1038/nature21029
27 Monje, M. & Iwasaki, A. The neurobiology of long COVID. Neuron 110, 3484-3496 (2022). doi.org:10.1016/j.neuron.2022.10.006
28 Volbracht, C., van Beek, J., Zhu, C., Blomgren, K. & Leist, M. Neuroprotective properties of memantine in different in vitro and in vivo models of excitotoxicity. Eur J Neurosci 23, 2611-2622 (2006). doi.org:10.1111/j.1460-9568.2006.04787.x
29 Wu, H. M. et al. Novel neuroprotective mechanisms of memantine: increase in neurotrophic factor release from astroglia and anti-inflammation by preventing microglial activation. Neuropsychopharmacology 34, 2344-2357 (2009). doi.org:10.1038/npp.2009.64
30 Brison, E., Jacomy, H., Desforges, M. & Talbot, P. J. Novel treatment with neuroprotective and antiviral properties against a neuroinvasive human respiratory virus. J Virol 88, 1548-1563 (2014). doi.org:10.1128/JVI.02972-13
31 Rogawski, M. A. & Wenk, G. L. The neuropharmacological basis for the use of memantine in the treatment of Alzheimer's disease. CNS Drug Rev 9, 275-308 (2003). doi.org:10.1111/j.1527-3458.2003.tb00254.x
32 Mony, L., Kew, J. N., Gunthorpe, M. J. & Paoletti, P. Allosteric modulators of NR2B-containing NMDA receptors: molecular mechanisms and therapeutic potential. Br J Pharmacol 157, 1301-1317 (2009). doi.org:10.1111/j.1476-5381.2009.00304.x
33 Fischler, P. V., Soyka, M., Seifritz, E. & Mutschler, J. Off-label and investigational drugs in the treatment of alcohol use disorder: A critical review. Front Pharmacol 13, 927703 (2022). doi.org:10.3389/fphar.2022.927703
34 Paoletti, P. & Neyton, J. NMDA receptor subunits: function and pharmacology. Curr Opin Pharmacol 7, 39-47 (2007). doi.org:10.1016/j.coph.2006.08.011
35 Haroon, E., Miller, A. H. & Sanacora, G. Inflammation, Glutamate, and Glia: A Trio of Trouble in Mood Disorders. Neuropsychopharmacology 42, 193-215 (2017). doi.org:10.1038/npp.2016.199
36 Iovino, L., Tremblay, M. E. & Civiero, L. Glutamate-induced excitotoxicity in Parkinson's disease: The role of glial cells. J Pharmacol Sci 144, 151-164 (2020). doi.org:10.1016/j.jphs.2020.07.011
37 NIH. Guidelines for Endpoints in Animal Studies. (National Institute of Health, nih.gov, 2022).
38 Crunfli, F. et al. Morphological, cellular, and molecular basis of brain infection in COVID-19 patients. Proc Natl Acad Sci USA 119, e2200960119 (2022). doi.org:10.1073/pnas.2200960119
39 Politi, L. S., Salsano, E. & Grimaldi, M. Magnetic Resonance Imaging Alteration of the Brain in a Patient With Coronavirus Disease 2019 (COVID-19) and Anosmia. JAMA Neurol 77, 1028-1029 (2020). doi.org:10.1001/jamaneurol.2020.2125

40 Garcia-Cabezas, M. A., John, Y. J., Barbas, H. & Zikopoulos, B. Distinction of Neurons, Glia and Endothelial Cells in the Cerebral Cortex: An Algorithm Based on Cytological Features. Front Neuroanat 10, 107 (2016). doi.org:10.3389/fnana.2016.00107
41 Andrews, M. G. et al. Tropism of SARS-CoV-2 for human cortical astrocytes. Proc Natl Acad Sci USA 119, e2122236119 (2022). doi.org:10.1073/pnas.2122236119
42 Ikenari, T., Kurata, H., Satoh, T., Hata, Y. & Mori, T. Evaluation of Fluoro-Jade C Staining: Specificity and Application to Damaged Immature Neuronal Cells in the Normal and Injured Mouse Brain. Neuroscience 425, 146-156 (2020). doi.org:10.1016/j.neuroscience.2019.11.029
43 Boroujeni, M. E. et al. Inflammatory Response Leads to Neuronal Death in Human Post-Mortem Cerebral Cortex in Patients with COVID-19. ACS Chem Neurosci 12, 2143-2150 (2021). doi.org:10.1021/acschemneuro.1c00111
44 Reichard, R. R. et al. Neuropathology of COVID-19: a spectrum of vascular and acute disseminated encephalomyelitis (ADEM)-like pathology. Acta Neuropathol 140, 1-6 (2020). doi.org:10.1007/s00401-020-02166-2
45 Nabizadeh, F. et al. Autoimmune encephalitis associated with COVID-19: A systematic review. Mult Scler Relat Disord 62, 103795 (2022). doi.org:10.1016/j.msard.2022.103795
46 Naidu, K. & Tayler, R. Anti N-Methyl-D-Aspartate receptor antibody associated Acute Demyelinating Encephalomyelitis in a patient with COVID-19: a case report. J Med Case Rep 17, 247 (2023). doi.org:10.1186/s13256-023-03979-x
47 Lee, H. et al. Anti-N-methyl-D-aspartate receptor encephalitis after coronavirus disease 2019: A case report and literature review. Medicine (Baltimore) 101, e30464 (2022). doi.org:10.1097/MD.0000000000030464
48 Giovannoni, F. & Quintana, F. J. The Role of Astrocytes in CNS Inflammation. Trends Immunol 41, 805-819 (2020). doi.org:10.1016/j.it.2020.07.007
49 Sofroniew, M. V. Astrogliosis. Cold Spring Harb Perspect Biol 7, a020420 (2014). doi.org:10.1101/cshperspect.a020420
50 Rothstein, J. D. et al. Knockout of glutamate transporters reveals a major role for astroglial transport in excitotoxicity and clearance of glutamate. Neuron 16, 675-686 (1996). doi.org:10.1016/s0896-6273 (00) 80086-0
51 Hardin-Pouzet, H. et al. Glutamate metabolism is down-regulated in astrocytes during experimental allergic encephalomyelitis. Glia 20, 79-85 (1997). doi.org:10.1002/(sici) 1098-1136 (199705) 20:1<79::aid-glia8>3.0.co;2-0
52 Werner, P., Pitt, D. & Raine, C. S. Multiple sclerosis: altered glutamate homeostasis in lesions correlates with oligodendrocyte and axonal damage. Ann Neurol 50, 169-180 (2001). doi.org:10.1002/ana.1077
53 Olney, J. W. et al. NMDA antagonist neurotoxicity: mechanism and prevention. Science 254, 1515-1518 (1991). doi.org:10.1126/science.1835799
54 Olney, J. W., Labruyere, J. & Price, M. T. Pathological changes induced in cerebrocortical neurons by phencyclidine and related drugs. Science 244, 1360-1362 (1989). doi.org:10.1126/science.2660263
55 Jordao, M. J. C. et al. Single-cell profiling identifies myeloid cell subsets with distinct fates during neuroinflammation. Science 363 (2019). doi.org:10.1126/science.aat7554
56 Wolf, S. A., Boddeke, H. W. & Kettenmann, H. Microglia in Physiology and Disease. Annu Rev Physiol 79, 619-643 (2017). doi.org:10.1146/annurev-physiol-022516-034406
57 Woodburn, S. C., Bollinger, J. L. & Wohleb, E. S. The semantics of microglia activation: neuroinflammation, homeostasis, and stress. J Neuroinflammation 18, 258 (2021). doi.org:10.1186/s12974-021-02309-6
58 Lier, J., Streit, W. J. & Bechmann, I. Beyond Activation: Characterizing Microglial Functional Phenotypes. Cells 10 (2021). doi.org:10.3390/cells10092236
59 Sun, M. et al. Microglia-Astrocyte Interaction in Neural Development and Neural Pathogenesis. Cells 12 (2023). doi.org:10.3390/cells12151942
60 Matsudaira, T. & Prinz, M. Life and death of microglia: Mechanisms governing microglial states and fates. Immunol Lett 245, 51-60 (2022). doi.org:10.1016/j.imlet.2022.04.001
61 He, X. et al. NLRP3-dependent pyroptosis is required for HIV-1 gp120-induced neuropathology. Cell Mol Immunol 17, 283-299 (2020). doi.org:10.1038/s41423-019-0260-y
62 Hu, X. et al. Herpes Simplex Virus 1 Induces Microglia Gasdermin D-Dependent Pyroptosis Through Activating the NLR Family Pyrin Domain Containing 3 Inflammasome. Front Microbiol 13, 838808 (2022). doi.org:10.3389/fmicb.2022.838808
63 Zhou, T. et al. Microglial debris is cleared by astrocytes via C4b-facilitated phagocytosis and degraded via RUBICON-dependent noncanonical autophagy in mice. Nat Commun 13, 6233 (2022). doi.org:10.1038/s41467-022-33932-3
64 Konishi, H. et al. Astrocytic phagocytosis is a compensatory mechanism for microglial dysfunction. EMBO J 39, e104464 (2020). doi.org:10.15252/embj.2020104464
65 Jo, M. et al. Astrocytic Orosomucoid-2 Modulates Microglial Activation and Neuroinflammation. J Neurosci 37, 2878-2894 (2017). doi.org:10.1523/JNEUROSCI.2534-16.2017

We claim:

1. A method for preventing a viral induced neuropathology in a subject comprising prophylactic administration of at least one N-methyl-D-aspartic acid receptor (NMDAR) antagonist to the subject.

2. The method of claim 1, wherein the at least one NMDAR antagonist is selected from memantine, ketamine, ifenprodil, donepezil, amantadine, atomoxetine, agmatine, dextrallorphan, dextromethorphan, dextrophan, dizocilpine, neramexane, remacemide, eliprodil, selfotel, or combinations thereof.

3. The method of claim 1, wherein the at least one NMDAR includes memantine.

4. The method of claim 2, further comprising administration with a nicotinic acetylcholine receptor (nAChR) antagonist, an anti-nicotinic, an anti-cholinergic, or a combination thereof.

5. The method of claim 2, further comprising administration with an anti-viral therapy.

6. The method of claim 2, further comprising administration with a systemic steroid selected from the group consisting of dexamethasone, hydrocortisone, methylprednisolone, prednisone, or combinations thereof.

7. The method of claim 2, further comprising administration with nicotine.

8. The method of claim 2, wherein the viral induced neuropathology is caused by a measles virus, a coronavirus, an enterovirus, a adenovirus, an arbovirus, an Arenavirdae, a Bornavirdae, a Flavivirdae, a Hepadnavirdae, a Herpesvirdae, human immunodeficiency virus, human T-lymphotropic virus type I, a paramyxovirdae, a Picornavirdae, a Rhabdovirdae, a Togavirdae, and/or an influenza virus.

9. The method of claim 8, wherein the virus is SARS-CoV-1, MERS-CoV, and/or SARS-CoV-2 and/or its variants.

10. The method of claim 2, wherein the subject is vulnerable to a viral infection.

11. The method of claim 2, wherein the at least one NMDAR antagonist is administered orally, intravenously, by infusion, transdermally, sublingually, intramuscularly, by inhalation, rectally, vaginally, subcutaneously, opthalmically, buccally, nasally, enterally, topically, intrathecally, or combinations thereof.

12. The method of claim 2, wherein the NMDAR antagonist is administered with a vehicle, an excipient, and/or a pharmaceutically acceptable carrier.

13. A method for treating a viral induced neuropathology in a subject comprising prophylactic administration of at least one N-methyl-D-aspartic acid receptor (NMDAR) antagonist to the subject.

14. The method of claim 13, wherein the at least one NMDAR antagonist is selected from memantine, ketamine, ifenprodil, donepezil, amantadine, atomoxetine, agmatine, dextrallorphan, dextromethorphan, dextrophan, dizocilpine, neramexane, remacemide, eliprodil, selfotel, or combinations thereof.

15. The method of claim 13, wherein the at least one NMDAR includes memantine.

16. The method of claim 14, further comprising administration with a nAChR antagonist, an anti-nicotinic, an anti-cholinergic, or a combination thereof.

17. The method of claim 14, further comprising administration with an anti-viral therapy.

18. The method of claim 14, further comprising administration with a systemic steroid selected from the group consisting of dexamethasone, hydrocortisone, methylprednisolone, prednisone, or combinations thereof.

19. The method of claim 14, wherein the viral induced neuropathology is caused by a measles virus, a coronavirus, an enterovirus, a adenovirus, an arbovirus, an Arenavirdae, a Bornavirdae, a Flavivirdae, a Hepadnavirdae, a Herpesvirdae, human immunodeficiency virus, human T-lymphotropic virus type I, a paramyxovirdae, a Picornavirdae, a Rhabdovirdae, a Togavirdae, and/or an influenza virus.

20. The method of claim 19, wherein the virus is SARS-CoV-1, MERS-CoV, and/or SARS-CoV-2 and/or its variants.

21. The method of claim 14, wherein the subject is vulnerable to a viral infection.

22. The method of claim 14, wherein the at least one NMDAR antagonist is administered orally, intravenously, by infusion, transdermally, sublingually, intramuscularly, by inhalation, rectally, vaginally, subcutaneously, opthalmically, buccally, nasally, enterally, topically, intrathecally, or combinations thereof.

23. The method of claim 14, wherein the NMDAR antagonist is administered with a vehicle, an excipient, and/or a pharmaceutically acceptable carrier.
